(19) United States
(12) Patent Application Publication
(10) Pub. No.: US 2025/0052700 A1
(43) Pub. Date: Feb. 13, 2025

(54) METHOD OF ANALYSING A GEOLOGICAL SAMPLE

(71) Applicant: ADAPTIX LTD, Begbroke (GB)

(72) Inventor: Mark Evans, Witney (GB)

(21) Appl. No.: 18/927,650

(22) Filed: Oct. 25, 2024

Related U.S. Application Data
(63) Continuation of application No. PCT/GB2023/051002, filed on Apr. 13, 2023.

(30) Foreign Application Priority Data
Apr. 25, 2022 (GB) 2205992.7

(51) Int. Cl. G01N 23/04; G01N 21/64; G01N 23/083; G01N 33/24

(52) U.S. Cl. CPC G01N 23/04; G01N 21/6456; G01N 23/083; G01N 33/24; G01N 2223/401; G01N 2223/616

(57) ABSTRACT

A geological sample is provided to analysis equipment including a first X-ray emitter and a digital X-ray detector. The actions of activating the first X-ray emitter, located in a first position; activating a second X-ray emitter, located in a different position to the first X-ray emitter, or moving the first X-ray emitter to a different position and activating it; repeating the imaging step as required to analyse the core, each time using a different X-ray emitter, or the first X-ray emitter located in a different position; wherein the location of the digital detector, relative to the sample, remains stationary during the imaging steps; processing the digital signals provided by the digital detector to create digital X-ray images; processing the digital signals to determine compounds within, and porosity of, the sample by means of relative absorption at different X-ray energy levels; and processing the digital X-ray images are performed.

### RELATED APPLICATIONS

This application claims the benefit under 35 U.S.C. § 120, and is a continuation, of co-pending International Application PCT/GB2023/051002, filed Apr. 13, 2023 and designating the US, which claims priority to GB Application 2205992.7, filed Apr. 25, 2022, such GB Applications also being claimed priority to under 35 U.S.C. § 119. These GB and International applications are incorporated by reference herein in their entireties.

### FIELD

The present invention relates generally to a method of analysing a geological sample and apparatus arranged to perform the method, and finds particular, although not exclusive, utility in the analysis of geological cores to identify hydrocarbons and/or porosity.

### BACKGROUND

Geological core samples are extracted from the ground from a well. They can be extracted from depths of several thousand meters. When the decision has been made to extract a core, regular operations have to be stopped and the well stabilized, while the core is pulled up out of the ground with special equipment. Most of the cores are up to 27 meters long, but are often cut into meter-long sections with a diameter of ˜10 cm for transport.

Often sections of the core are sent to laboratories where labour intensive analysis is undertaken which can take three or four days. Computed tomography (CT) methods are known for analysing aspects of the cores. However, the equipment required to undertake CT analysis is relatively large and complex which makes it not readily installable or usable at well head locations.

### SUMMARY

It is desirable to have a method of geological sample analysis which reduces the need to transport the samples, which reduces the time taken to analyse them, and which reduces the level of skills required to analyse them.

In a first aspect, the present invention provides a method of analysing a geological sample comprising the steps of:
a) providing a geological sample and analysis equipment, said equipment including a first X-ray emitter and a digital X-ray detector;
b) activating the first X-ray emitter, located in a first position, and receiving the emitted X-rays at the digital X-ray detector;
c) activating a second X-ray emitter, located in a different position to the first X-ray emitter, or moving the first X-ray emitter to a different position and activating it, and receiving the emitted X-rays at the digital X-ray detector;
d) repeating step c) as required to analyse the core, each time using a different X-ray emitter, or the first X-ray emitter located in a different position;
e) wherein the location of the digital detector, relative to the sample, remains stationary during steps b) to d)
f) processing the digital signals provided by the digital detector to create digital X-ray images of the sample;
g) processing the digital signals provided by the digital detector to determine compounds within, and porosity of, the sample by means of relative absorption at different X-ray energy levels;
h) processing the digital X-ray images of the sample to create a digital tomosynthesis 3D X-ray image model of the sample, said model including a means of indicating the different compounds, and porosity of the sample, and their locations in the sample.

In one example, the method uses a single X-ray source mounted on a moving stage which can move in two dimensions, to allow multiple images of the same portion of the sample to be formed, from multiple directions, to allow the reconstruction of a 3D model of the sample using partial sweep computed tomography.

In another example, the method uses an X-ray emitter array, the array remaining stationary relative to the sample and digital detector in use, such that different X-ray emitters in the array are actuated sequentially to allow multiple images of the same portion of the sample to be formed, from multiple directions, to allow the reconstruction of a 3D model of the sample using digital tomosynthesis (‘DT’).

It is expected that the X-rays may be emitted at approximately 45 different positions.

It is expected that X-rays of multiple energy levels may be emitted simultaneously at each instance. For instance, energy levels from 0 to 60 kV may be employed. The different energy levels may be absorbed by different compounds (or elements) thus aiding the identification of the composition of the core.

The geological sample may be a geological core.

The method may further comprise the steps of:
i) illuminating the sample with ultraviolet light and using a digital camera to detect any fluorescence as a result;
j) processing the digital signals provided by the digital camera to provide data relating to any areas of fluorescence;
k) adding said data to said digital 3D X-ray image model to visually show said areas on said model.

The ultraviolet light wavelength may be selected such that hydrocarbons in the sample fluoresce. The method may include the step of darkening the immediate vicinity of the sample so as to promote the detection of fluorescence.

Step g) of the method may use the method of K-edge subtraction to determine the compounds within the sample.

The method may further comprise the steps of:
l) moving the sample relative to the at least one X-ray emitter and digital X-ray detector;
m) repeating the steps to analyse a different portion of the sample.

This step allows for samples which are larger (or just longer) than the apparatus can image in one position. The sample is moved and then the next portion is imaged as before. The processor may be arranged to stitch together the images from each sample position so as to create a single model.

The method may further comprise the step of comparing the data, such as the 3D images, acquired by the method with data acquired from other samples. The comparison may be algorithmic, and use machine learning to identify samples having similar characteristics, such as porosity, and distribution of identified compounds. In this way, if certain characteristics of cores are determined by a human expert reviewing the images, as being likely for hydrocarbon recovery, images of other cores may be compared by computer to find similar examples, thus increasing the speed of image assessment.

In a second aspect, the invention provides a geological sample analysis apparatus comprising at least one X-ray emitter, a digital X-ray detector, and a processor arranged to operate in accordance with any of the methods of the first aspect.

The geological sample analysis apparatus may further comprise an ultraviolet light source and a digital camera.

The geological sample analysis apparatus may further comprise a sample support and a motor for moving the sample relative to the at least one X-ray emitter. This relates to the method step which allows for samples which are larger (or just longer) than the apparatus can image with the sample in a single position. The sample is moved and then the next portion is imaged as before.

The method and apparatus allow for the analysis of geological samples to be undertaken at the well head without the need for their transportation to a laboratory. This is because the apparatus is relatively small and portable and may be automated. This is due to the method which does not need to move the X-ray detector and the X-ray emitter relative to the sample during imaging, as is the case with traditional CT equipment and methods.

It is also due to the X-ray power levels being of the order of 60 kV, which are substantially lower than CT equipment and methods, thus allowing relatively simple and low cost shielding to be used to protect operators. This reduces the size, weight and complexity of the apparatus and allows for simpler methods of operation.

The X-ray detector has multi-spectral capability, and the image reconstruction allows each voxel to be characterized as to the compounds within the voxel by reference to the relative absorption at a number of different X-ray energies. The location of each voxel is identified such that a 3D map of the core and its constituent components is provided.

This method and apparatus differs from a multi-spectral Computed Tomography (‘CT’) scanner as there is no need to move the detector, reducing cost; there is no need to rotate both the source and the detector around the subject, reducing cost; the system allows a fast acquisition (<1 minute), whereas a micro-CT scanner would typically take hours (sometimes 10s of hours); relative to CT apparatus and method, the reduced flux of the system, combined with the small size, allows either a fully-shielded system (which does not require a specialist, and expensive, shielded room) which allows the system to be easily deployed at low cost (relative to a CT) allowing forward deployment at the well head, or an even lighter unshielded system to be forward deployed by one person and be used in an open environment at a safe distance of just over 5 meters; relative to CT, the x-y resolution is better, and (as has been seen in medical applications) the ability to identify porosity, and, in conjunction with image processing techniques, the ability to provide a metric for porosity is viable. In this regard, the model may show areas of the sample having similar levels of porosity, or areas of the sample with porosity above a threshold level.

### BRIEF DESCRIPTION OF THE ACCOMPANYING DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference FIGURES quoted below refer to the attached drawings.

FIG. 1 is a schematic diagram of an X-ray apparatus.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other. For instance, wireless connectivity is contemplated.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, FIGURE, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

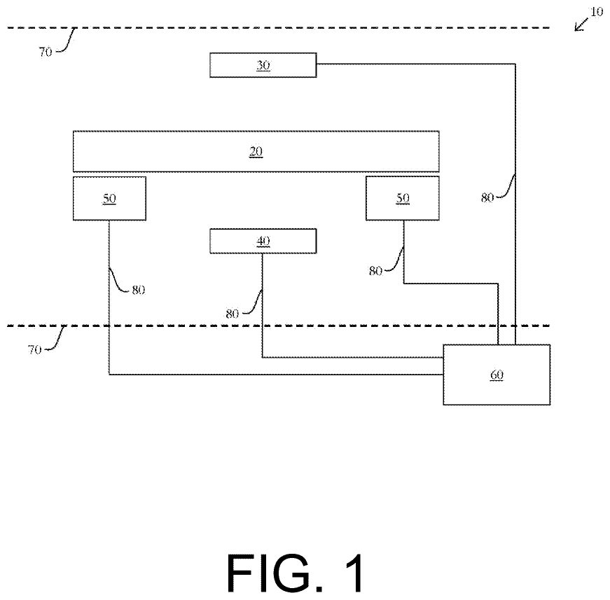

FIG. 1 shows X-ray apparatus 10 comprising an X-ray emitter 30 and an opposing X-ray detector 40. A geological core sample 20 is shown located between the emitter and detector such that it may be subjected to X-ray to be imaged.

The sample 20 is supported by supports 50 located at either end. However, the support may extend along the entire length and/or at other portions of the sample 20. These supports 50 may be moved to move the sample relative to the emitter and/or detector.

The various components 30, 40, 50 are connected to a processor 60 via means of communication 80 to control them and/or receive data therefrom.

Shielding 70 is indicatively shown above and below the apparatus 10. This may take the form of known X-ray shielding and be arranged in other locations around the apparatus 10.

1. A method of analysing a geological sample comprising the steps of:
a) providing a geological sample and analysis equipment, said equipment including a first X-ray emitter and a digital X-ray detector;
b) activating the first X-ray emitter, located in a first position, and receiving the emitted X-rays at the digital X-ray detector;
c) activating a second X-ray emitter, located in a different position to the first X-ray emitter, or moving the first X-ray emitter to a different position and activating it, and receiving the emitted X-rays at the digital X-ray detector;
d) repeating step c) as required to analyse the core, each time using a different X-ray emitter, or the first X-ray emitter located in a different position;
e) wherein the location of the digital detector, relative to the sample, remains stationary during steps b) to d)
f) processing the digital signals provided by the digital detector to create digital X-ray images of the sample;
g) processing the digital signals provided by the digital detector to determine compounds within, and porosity of, the sample by determining relative absorption at different X-ray energy levels;
h) processing the digital X-ray images of the sample to create a digital tomosynthesis 3D X-ray image model of the sample, said indicating the different compounds, and porosity of the sample, and their locations in the sample.

2. The method of claim 1, wherein the geological sample is a geological core.

3. The method of claim 1, further comprising the steps of:
i) illuminating the sample with ultraviolet light and using a digital camera to detect any fluorescence as a result;
j) processing the digital signals provided by the digital camera to provide data relating to any areas of fluorescence;
k) adding said data to said digital 3D X-ray image model to visually show said areas on said model.

4. The method of claim 3, wherein the ultraviolet light wavelength is selected such that hydrocarbons in the sample fluoresce.

5. The method of claim 1, wherein step g) uses the method of K-edge subtraction to determine the compounds within the sample.

6. The method of claim 1, further comprising the steps of:
l) moving the sample relative to the at least one X-ray emitter and digital X-ray detector;
m) repeating the steps to analyse a different portion of the sample.

7. A geological sample analysis apparatus comprising at least one X-ray emitter, a digital X-ray detector, and a processor arranged to operate in accordance with the method of claim 1.

8. The geological sample analysis apparatus of claim 7, further comprising an ultraviolet light source and a digital camera.

9. The geological sample analysis apparatus of claim 7, further comprising a sample support and a motor for moving the sample relative to the at least one X-ray emitter.
